(19) United States
(12) Patent Application Publication
(10) Pub. No.: US 2004/0268431 A1
(43) Pub. Date: Dec. 30, 2004

(54) Transgenic plant products comprising human granulocyte colony-stimulating factor and method for preparing the same

(75) Inventors: Sai Ming Samuel Sun, Honolulu, HI (US); Ming Chiu Fung, Hong Kong (CN); Hon Ming Lam, Hong Kong (CN)

Correspondence Address: MERCHANT & GOULD PC, P.O. BOX 2903, MINNEAPOLIS, MN, 55402-0903, US

(73) Assignee: THE CHINESE UNIVERSITY OF HONG KONG, Hong Kong (CN)

(21) Appl. No.: 10/611,226

(22) Filed: Jun. 30, 2003

(51) Int. Cl. C12N 15/82; C12N 15/87; C12N 15/09; C12N 15/63; C07K 14/00

(52) U.S. Cl. 800/278; 435/320.1; 800/294; 800/288; 530/350; 435/468; 800/298

(57) ABSTRACT

The present invention is to provide a recombinant construct for transforming a plant comprising a DNA sequence encoding a recombinant human cytokine and a promoter capable of directing the expression of the recombinant human cytokine in the plant. The present invention is also to provide a method for constructing a transgenic plant, comprising the steps of transforming a plant cell with a recombinant construct of the invention, and regenerating the transgenic plant from the plant cell to produce recombinant human cytokine, for example, human granulocyte colony stimulating factor (hG-CSF), in seeds of the transgenic plant. The plant production method of the invention thus has a promising potential to mass-produce some of the most expensive biopharmaceuticals of restricted availability in a much cheaper way, which has high economic value for disease therapy, diagnosis and prevention, and is more accessible to the less affluent countries.

### FIELD OF THE INVENTION

The present invention is directed to high expression of foreign genes in plants, in particular to a recombinant construct comprising human granulocyte colony-stimulating factor and to transgenic plants which contain the same.

### BACKGROUND OF THE INVENTION

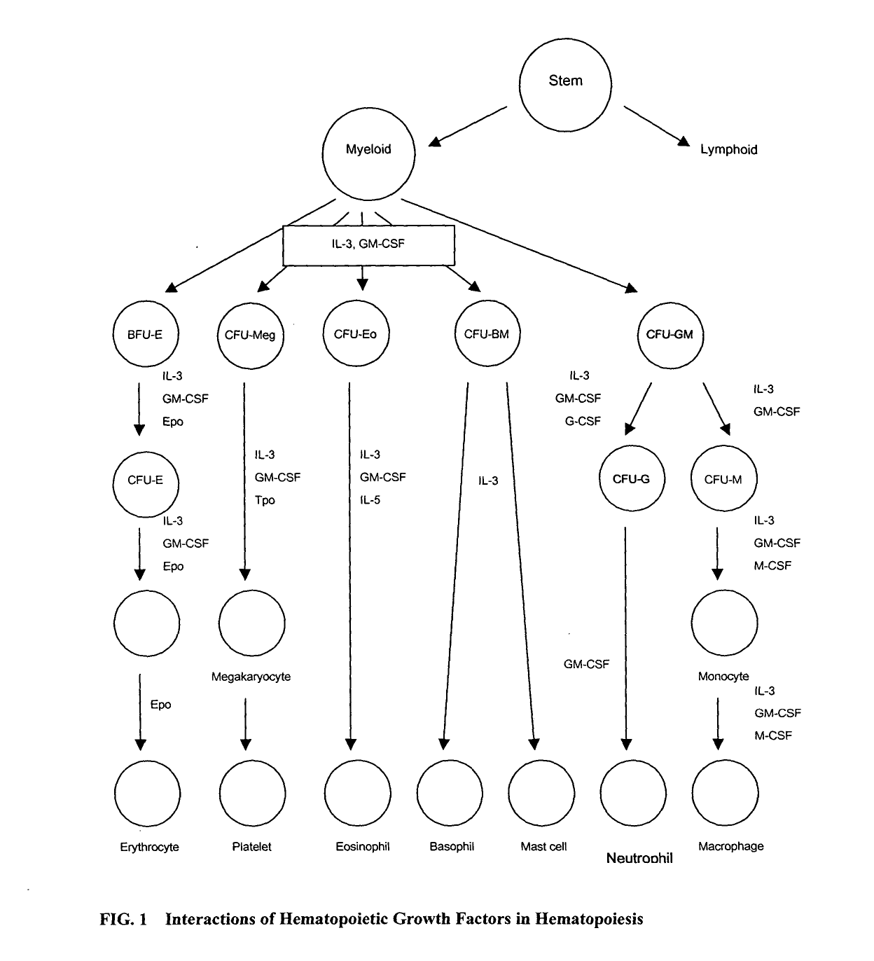

Human granulocyte colony-stimulating factor (hG-CSF) is one of the colony stimulating factors (CSFs) or hematopoietic growth factors. It has been known that hG-CSF can be produced by primary bone marrow stromal cells, macrophages, fibroblasts and endothelial cells, upon various kinds of stimulations such as infections and inflammations. (Metcalf, D. and Nicola, N. A. 1985, Synthesis by Mouse Peritoneal Cells of G-CSF, the Differentiation Inducer for Myeloid Leukemia Cells: Stimulation by Endotoxin, M-CSF and Multi-CSF, Leuk. Res. 9, 35-50; Broudy, V. C., Kaushansky, K., Harlan, J. M. and Adamson, J. W. 1987, Interleukin 1 Stimulates Human Endothelial Cells to Produce Granulocyte-Macrophage Colony-Stimulating Factor and Granulocyte Colony-Stimulating Factor, J. Immunol. 139, 464-468; Kaushansky, K, Lin, N. and Adamson, J. W. 1988, Interleukin 1 Stimulates Fibroblasts to Synthesize Granulocyte-Macrophage and Granulocyte Colony-Stimulating Factors. Mechanism for the Hematopoietic Response to Inflammation, J. Clin. Invest. 81, 92-97; Vellenga, E., Rambaldi, A., Ernst, T. J., Ostapovicz, D. and Griffin, J. D. 1988, Independent Regulation of M-CSF and G-CSF Gene Expression in Human Monocytes, Blood 71, 1529-1532). As shown in FIG. 1, hG-CSF can specifically stimulate the proliferation and differentiation of a CFU-GM (colony forming units-granulocyte/monocyte) and CFU-G (granulocyte) into a mature neutrophil, which is one kind of white blood cells or leukocytes, and protect human body by ingestion and killing of invading bacteria and other microorganisms (Palmblad, J. 1984, The Role of Granulocytes in Inflammation, Scand. J. Rheum. 13, 163-172).

However, the half-life of the neutrophil is short so that hG-CSF plays an essential role in both maintaining a basal level of the neutrophil in the body and greatly increasing the amount thereof during infection by regulating the production of the neutrophil from pluripotent stem cells in the bone marrow. Besides, hG-CSF can prolong the neutrophil survival (Williams, G. T., Smith, C. A., Spooncer, E., Dexter, T. M. and Taylor, D. R. 1990, Haemopoietic Colony Stimulating Factors Promote Cell Survival by Suppressing Apoptosis. Nature 343, 76-79.), increase its functional capacity (Kitagawa, S., You, A., Souza, L. M., Saito, M., Miura, Y. and Takaku, F. 1987, Recombinant Human Granulocyte Colony-Stimulating Factor Enhances Superoxide Release in Human Granulocytes Stimulated by the Chemotactic Peptide, Biochem. Biophys. Res. Commun. 144, 1143-1146; Yuo, A., Kitagawa, S., Ohsaka, A., Ohta, M., Miyazono, K., Okabe, T., Urabe, A., Saito, M. and Takaku, F. 1989, Recombinant Human Granulocyte Colony-Stimulating Factor as an Activator of Human Granulocytes: Potentiation of Responses Triggered by Receptor-Mediated Agonists and Stimulation of C3bi Receptor Expression and Adherence. Blood 74, 2144-2149; Yuo, A., Kitagawa, S., Ohsaka, A., Saito, M. and Takaku, F. 1990, Stimulation and Priming of Human Neutrophils by Granulocyte Colony-Stimulating Factor and Granulocyte-Macrophage Colony-Stimulating Factor: Qualitative and Quantitative Differences, Biochem. Biophys. Res. Commun. 171, 491-497), and stimulate the neutrophil mobilization from bone marrow into blood and tissues (Hattori, K., Orita, T., Oheda, M., Tamura, M. and Ono, M. 1996, Comparative Study of the Effects of Granulocyte Colony-Stimulating Factor and Granulocyte-Macrophage Colony-Stimulating Factor on Generation and Mobilization of Neutrophils in Cyclophosphamide-Treated Neutropenic Mice, In Vivo 10, 319-327). Therefore, hG-CSF plays an important role in protecting our bodies from bacterial, fungal and viral infections by regulating the production of mature and functional neutrophils. Administration of hG-CSF can reduce the duration of neutropenia and risks of various infections, which has a great benefit to many cancer patients after chemotherapy and radiotherapy. Moreover, hG-CSF also plays an essential role in treatment of neutropenia from other diseases and mobilization of hematopoietic stem cells. As a result, there is a very high demand of hG-CSF in clinical applications all over the world.

Meanwhile, the overall rhG-CSF for sale in all pharmaceutical companies was over US $2 billion in 2000. Nowadays, rhG-CSF is one of the top-selling pharmaceutical proteins and the best selling products on the anti-cancer drug market. In addition, hG-CSF therapy can in turn reduce the duration of hospitalization (25.3 days vs 29.8 days) and antibiotic therapy (14.5 days vs 18.6 days) with subsequent cost reduction to both hospitals and patients (Faulds, D., Lewis, N. J. W. and Milne, R. J. 1992, Recombinant Granulocyte Colony-Stimulating Factor (rG-CSF): Pharmacoeconomic Considerations in Chemotherapy-Induced Neutropenia, PharmacoEconomics 1, 231-249; Duncan, N., Hewetson, M., Atra, A., Dick, G. and Pinkerton, R. 1997, An Economic Evaluation of the Use of Granulocyte Colony-Stimulating Factor after Bone Marrow Transplantation in Children, PhamacoEconomics 11, 169-174). Due to these important and beneficial factors, the economic value of hG-CSF is very high and it is worth producing this pharmaceutical protein in large scale for clinical use.

Recent advancements in plant molecular biology and biotechnology have provided requisite tools to transform plants with foreign gene to produce biomolecules and heterologous proteins, such as lipids, carbohydrates, industrial enzymes and pharmaceutical proteins (Goddijn, O. J. M. and Pen, J. 1995, Plants as Bioreactors, Trends in Biotechnology 13, 379-387). The commercial potential of using transgenic plants as production systems is very high due to the unique and outstanding characteristics of the plants (Giddings, G., Allison, G., Brooks, D. and Carter, A. 2000, Transgenic Plants as Factories for Biopharmaceuticals, Nature Biotechnology 18, 1151-1155). Plant production systems are more economical compared to fermentation-based production systems. The production cost of transgenic plants is low, as plants only require water, soil, sunlight and some fertilizers for efficient growth while huge capital investments such as expensive fermenters, equipments and medium are needed in the fermentation-based production system (Goddijn, O. J. M. and Pen, J. 1995, Plants as Bioreactors, Trends in Biotechnology 13, 379-387). Moreover, for transgenic plants, the process of scale-up is simple, fast and inexpensive while scale-up in fermentation-based production is complex, time-consuming and expensive. According to Kusnadi et al. (Kusnadi, A. R., Nikolov, Z. L. and Howard, J. A. 1997, Production of recombinant proteins in transgenic plants: practical considerations, Biotechnology and Bioengineering 56, 473-484), the cost of producing recombinant proteins in plants has been estimated to be 10- to-50-fold lower than that in E. coli fermentation. Plant production systems can offer a promising potential to mass-produce some of the most expensive biopharmaceuticals of restricted availability, such as glucocerebrosidase, in a much economical way (Giddings, G., Allison, G., Brooks, D. and Carter, A. 2000, Transgenic Plants as Factories for Biopharmaceuticals, Nature Biotechnology 18, 1151-1155). However, scientists who tried to develop plants as bioreactors have encountered a major obstacle of a low yield of foreign proteins in transgenic plants.

Enhancing the transcription of a target protein encoding sequence through constructing chimeric genes with a strong and seed-specific promoter such as a phaseolin promoter to direct the expression of the target protein in plant seeds has been proved as an effective method to solve the problem in the art.

The promoter of the highly expressed seed-specific proteins such as phaseolin has been used for constructing the chimeric genes to transform plants to produce the target protein. As phaseolin is an abundant seed protein, the phaseolin promoter, which is seed-specific, is of great interest for transgenic expression of foreign proteins. Altenbach et al. (Altenbach, S. B., Pearson, K. W., Meeker, G., Staraci, L. C. and Sun, S. S. M. 1989, Enhancement of the Methionine Content of Seed Proteins by the Expression of a Chimeric Gene Encoding a Methionine-rich Protein in Transgenic Plants, Plant Molecular Biology 13, 513-522) demonstrated the transgenic expression of Brazil nut methionine-rich protein in tobacco using the phaseolin promoter as regulatory elements. An increase in methionine content, up to 30%, in transgenic tobacco seeds has been recorded as a result of strong expression of the phaseolin promoter.

Phaseolin is a group of polypeptides comprising the major seed storage glycoproteins of French bean (Phaseolus vulgaris L.). It accounts for about 50% of the total protein in the mature seed (Ma, Y. and Bliss, F. A. 1978, Seed Proteins of Common bean, Crop Sci. 17, 431-437). The protein consists of 3 subunits including α, β, and γ polypeptides of 51, 48 and 45.5 kD, respectively. Sun et al. (Sun, S. S. M., Mutschler, M. A., Bliss, F. A. and Hall, T. C. 1978, Protein Synthesis and Accumulation in Bean Cotyledons during Growth, Plant Physiology 61, 918-923.) demonstrated temporal accumulation of phaseolin during embryo development of the French bean. The three polypeptides are encoded by 16S mRNA species accumulating in developing cotyledon of 7 mm to 17-19 mm in length. Further studies on the β-phaseolin gene by Bustos et al. (Bustos, M. M., Guiltinan, M. J., Jordano, J., Begum, D., Kalkan, F. A. and Hall, T. C. 1989, Regulation of β-Glucuronidase Expression in Transgenic Tobacco Plants by an A/T-rich, cis-Acting Sequence Found Upstream of a French Bean β-Phaseolin Gene, Plant Cell 1, 839-853.) have shown the presence of multiple cis-acting elements around the −295 to +20 region of the phaseolin gene, which is responsible for the seed specific and temporal control of gene expression.

By electroporation, Agrobacterium tumefaciens may be transformed with a chimeric gene. The Agrobacterium GV3101/pMP90 (Koncz, C. and Schell, J. 1986, The Promoter of the TL-DNA Gene 5 Controls the Tissue-specific Expression of Chimeric Genes Carried by a Novel Type of Agrobacterium Binary Vector, Mol. Gen. Genet. 204, 383-396.) and Agrobacterium LBA4404/pAL4404 (Hoekema, A., Hirsch, P. R., Hooykaas, P. J. J. and Schilperoot, R. A. 1983, A Binary Plant Vector Strategy Based on Separation of vir- and T-region of the Agrobacterium tumefaciens Ti-plasmid, Nature 303, 179-180) were transformed into host plants Arabidopsis and tobacco through the known methods of vacuum infiltration (Bechtold, N., Ellis, J. and Pelletier, G. 1993, In planta Agrobacterium-mediated Gene Transfer by Infiltration of Adult Arabidopsis thaliana Plants, C. R. Acad. Sci. Paris, Life Sci. 316, 1194-1199) and leaf-disc technique (Fisher, D. K. and Guiltinan, M. J. 1995, Rapid, Efficient Production of Homozygous Transgenic Tobacco Plants with Agrobacterium tumefaciens: A Seed-to-Seed Protocol, Plant Mol. Bio. 13, 278-289), respectively.

### SUMMARY OF THE INVENTION

An object of the invention is to provide a recombinant construct for transforming a plant comprising a DNA sequence encoding a recombinant human cytokine and a promoter capable of directing the expression of the recombinant human cytokine in the plant.

In one embodiment of the invention, the human cytokine is a human granulocyte colony stimulating factor (hG-CSF).

Seed-specific promoters are preferably used in the invention. In one embodiment, the plant seed-specific promoter is derived from phaseolin.

In one preferred embodiment of the invention, the recombinant construct may further comprise sequence tag and cleavage site.

In another embodiment of the invention, the recombinant construct may further comprise a His-tag and an EK site.

In another embodiment of the invention, the recombinant construct may further comprise a signal peptide. A phaseolin signal peptide is preferably used in the invention.

Another object of the present invention is to provide a method for constructing a transgenic plant, comprising the steps of:

a) transforming a plant cell with a recombinant construct of the invention; and

b) regenerating the transgenic plant from the plant cell to produce recombinant human cytokine in seeds of the transgenic plant.

In the method of the invention, the plant cell may be transformed by an Agrobacterium system. In one embodiment of the invention, the Agrobacterium system is an Agrobacterium tumefaciens-Ti plasmid system.

In the method according to the present invention, the plant may be selected from Arabidopsis thaliana and tobacco.

In the method of the present invention, the plant cell may be transformed by vacuum infiltration of flowering buds for Arabidopsis thaliana or by infection of leaf disc explants for tobacco.

Another object of the present invention is to provide a transgenic plant comprising a recombinant human cytokine. In one embodiment of the method of the invention, the human cytokine used is a human granulocyte colony stimulating factor.

Still another object of the present invention is to provide a transgenic plant that comprises a target gene defined in this invention. In the transgenic plants of the invention, Arabidopsis thaliana and tobacco are preferable.

The present invention also provides a seed of the transgenic plant that is defined above. Cytokine hG-CSF has not been expressed in plants before. The work of the present invention makes a breakthrough in the art of genetically engineering proteins and produces them in a relatively large amount and high biological activity.

The present invention hereby provides a solution for enhancing translation efficiency by introducing the seed-specific promoter such as phaseolin promoter to construct a chemeric gene.

The method of plant production according to the invention has a promising potential to mass-produce some of the most expensive biopharmaceuticals of restricted availability in a much cheaper way and to make those biopharmaceuticals, which has high economic value for disease therapy, diagnosis and prevention, and is more accessible to less affluent developing countries.

### BRIEF DESCRIPTION OF THE INVENTION

FIG. 1 diagrammatically shows interactions of hematopoietic growth factors in hematopoiesis.

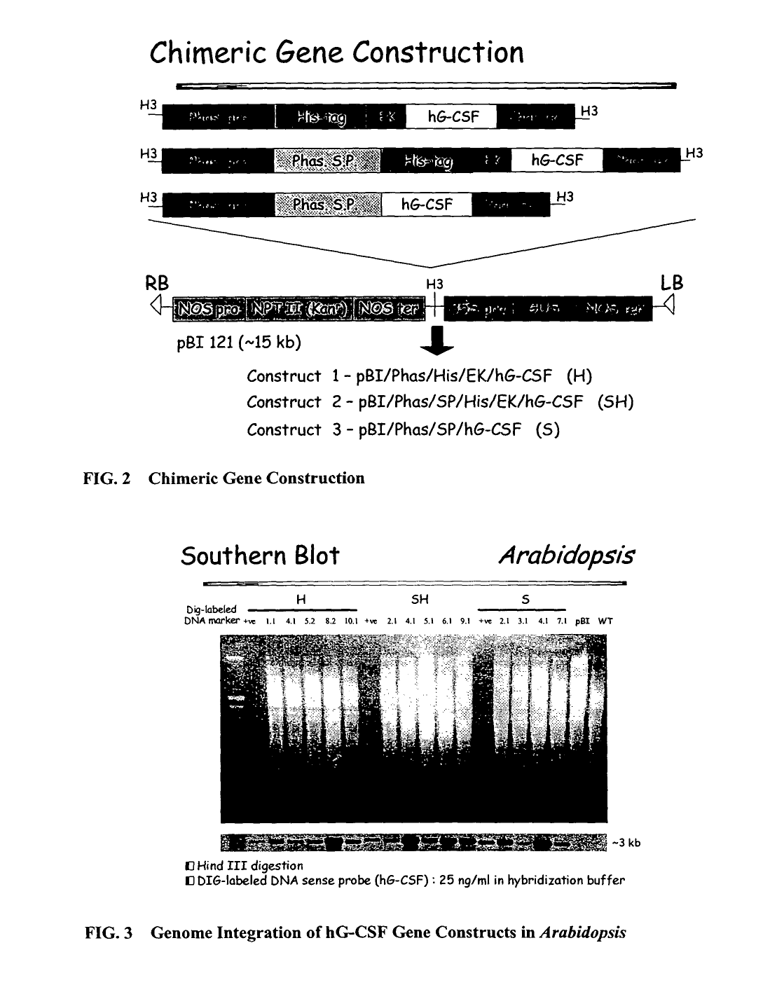

FIG. 2 shows construction of chimeric genes according to the invention.

FIG. 3 shows the result of Southern blot indicating genome integration of hG-CSF gene constructs in Arabidopsis according to the invention.

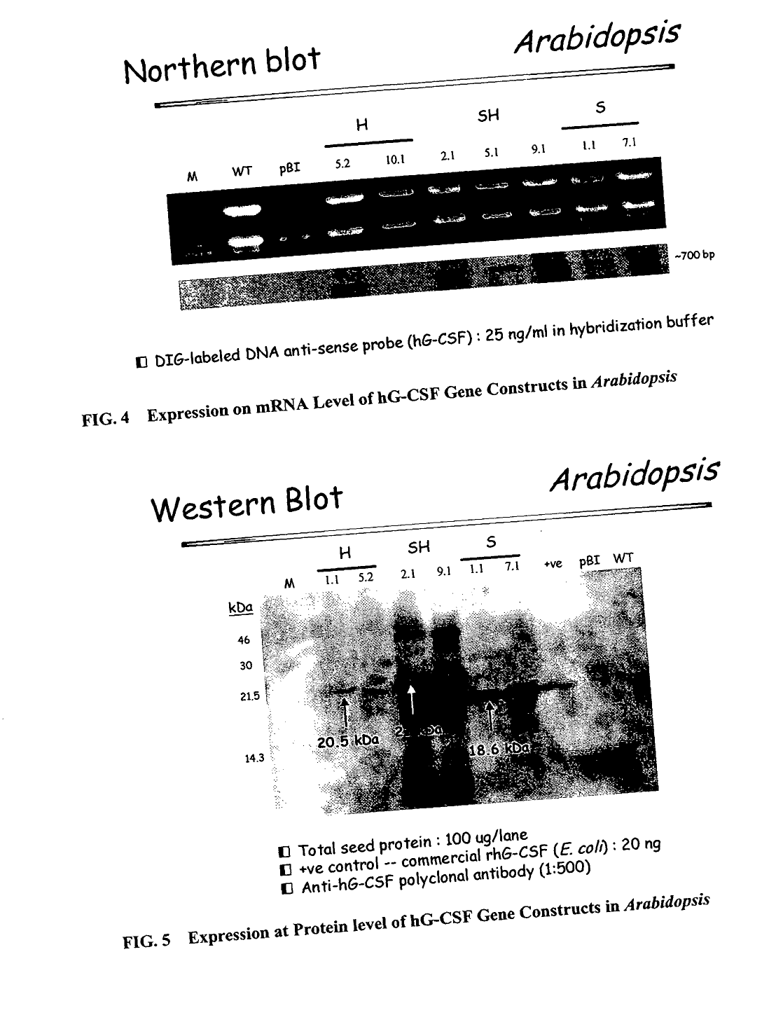

FIG. 4 shows the result of Northern blot indicating expression on MRNA level of hG-CSF gene constructs in Arabidopsis according to the invention.

FIG. 5 shows the result of Western blot indicating expression at protein level of hG-CSF gene constructs in Arabidopsis according to the invention.

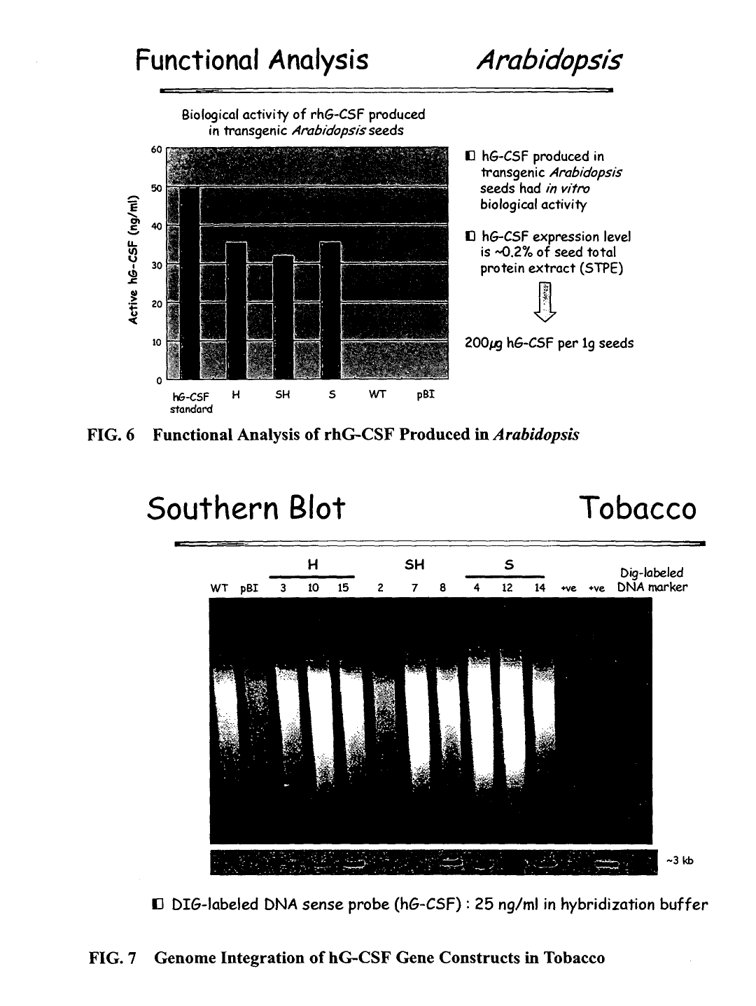

FIG. 6 shows functional analysis of rhG-CSF produced in Arabidopsis.

FIG. 7 shows the result of Southern blot indicating genome integration of hG-CSF gene constructs in tobacco according to the invention.

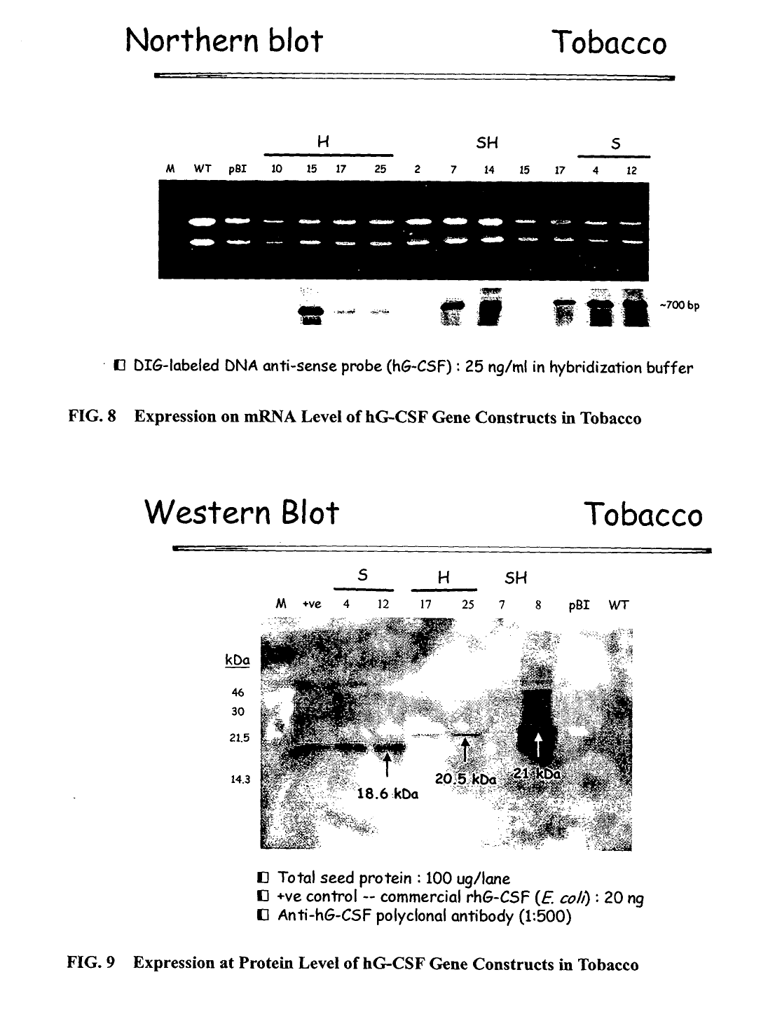

FIG. 8 shows the results of Northern blot indicating expression on MRNA level of hG-CSF gene constructs in tobacco according to the invention.

FIG. 9 shows the results of Western blot indicating expression at protein level of hG-CSF gene constructs in tobacco according to the invention.

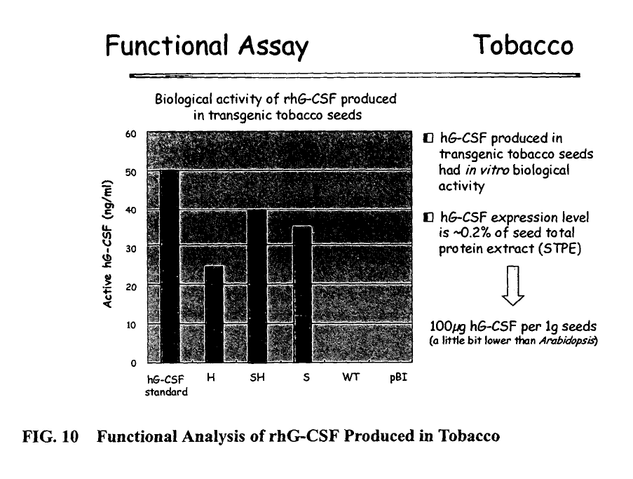

FIG. 10 shows functional analysis of rhG-CSF produced in tobacco according to the invention.

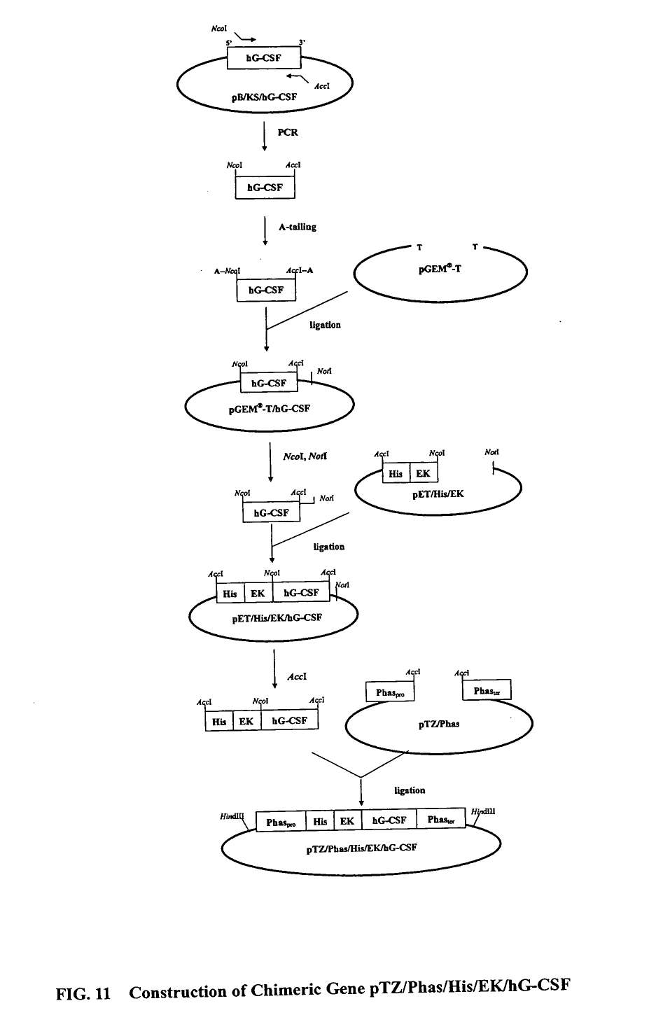

FIG. 11 shows construction of a chimeric gene pTZ/Phas/His/EK/hG-CSF according to the invention.

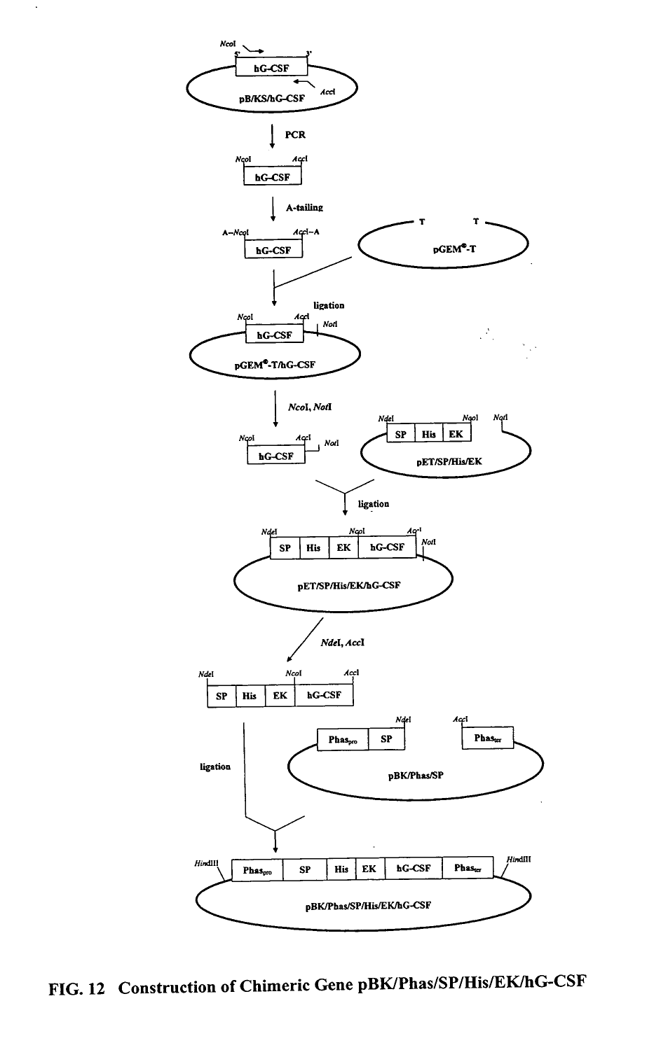

FIG. 12 shows construction of a chimeric gene pBK/Phas/SP/His/EK/hG-CSF according to the invention.

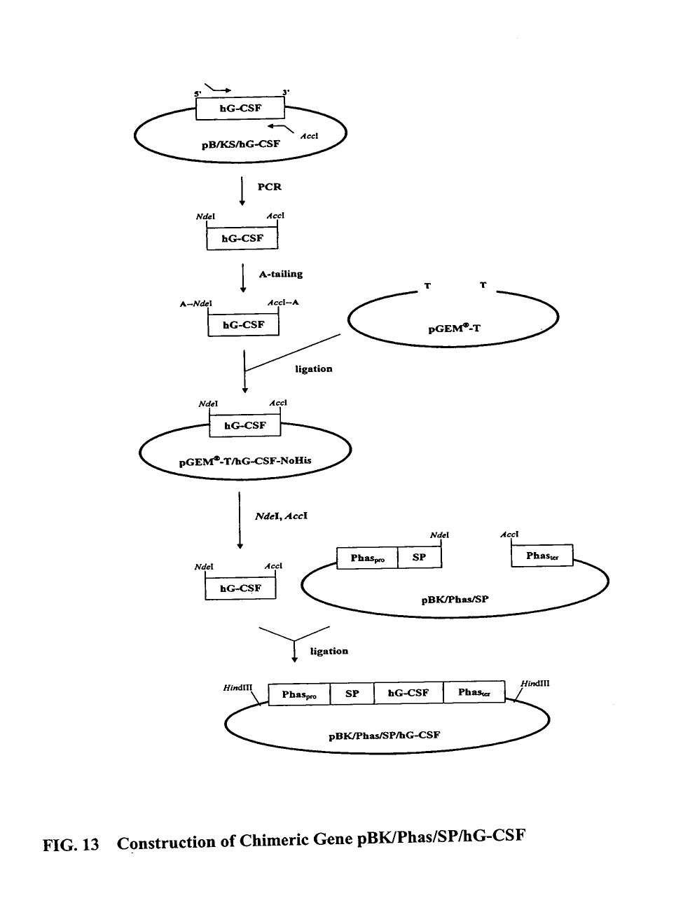

FIG. 13 shows construction of a chimeric gene pBK/Phas/SP/hG-CSF according to the invention.

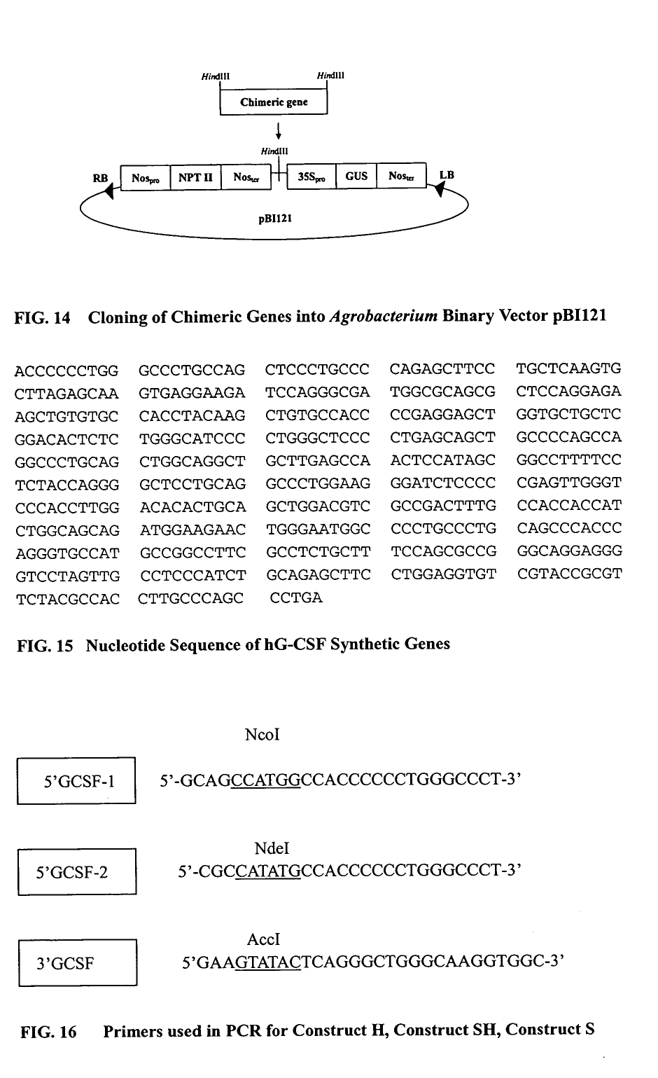

FIG. 14 shows cloning of chimeric genes into Agrobacterium binary vector pBI121 according to the invention.

FIG. 15 shows a nucleotide sequence of the hG-CSF synthetic gene according to the invention.

FIG. 16 show primers used in PCR for Construct H, Construct SH, Construct S.

### DETAILED DESCRIPTION OF THE INVENTION

As stated above, the objects of the invention have been fulfilled with a specific recombinant construct that comprises a strong seed-specific promoter to direct the expression of the target protein(s) in the seeds of transgenic plant.

In the present invention, the recombination construct comprises a strong seed-specific promoter, a plant seed-specific terminator and a DNA sequence encoding recombinant human cytokine.

In the present invention, those skilled in the art can easily select promoters or terminators specific to a plant seed that have been published. Promoters and terminators derived from phaseolin are preferably used in the invention.

It is well-known that the target gene must be amplified so as to have sufficient DNA for construction of enough chimeric genes. In the invention, a nucleotide sequence (525 bp) encoding the hG-CSF mature peptide (SEQ. ID No.1, FIG. 15) is first amplified by PCR using two specific primers, which can introduce a single restricted enzyme slicing site to two sides of the target gene for sub-cloning.

In the invention, the chimeric gene generally comprises a promoter, a target protein gene, and a terminator. Preferably, the promoter should be of high expression in plants and more preferably being stage-and-organ-specific. In the present invention, phaseolin is also preferably used as a seed-specific protein.

There are different kinds of sequences that can be fused to the target protein for subsequent affinity purification of the protein product, such as His-tag and S-tag. To remove these purification sequence tags from the final product, a specific enzyme cleavage site is frequently engineered between the sequence tag and the target protein, such as a eudokinase (EK) site. In this invention, a His-tag and an EK site are used for product purification purpose.

As shown in FIG. 2, three chimeric genes are constructed using a human G-CSF coding sequence and a French bean phaseolin promoter and terminator. These include construct H with a His-tag and an EK site, construct SH with a phaseolin signal peptide and a His-tag and an EK site, and construct S with a phaseolin signal peptide only.

Many vectors that are conventional in the art can be used to build a further construct in the invention. A binary vector, such as Agrobacterium binary vector that is the most popular vector for transforming plants, is preferably used in the invention. The Agrobacterium binary vector generally consists of the right border (RB) and left border (LB) of T-DNA, a neomycin phosphotransferase II (NPT II) selectable marker and a β-glucurondiase (GUS) screenable marker gene. RB and LB are used to transfer the DNA region between them to the genome of specific plants. NPT II gene is used to screen the plant transformants in a culture medium containing kanamycin while the GUS gene is used to confirm the transformants by enzyme assay. The chimeric gene in plasmids is excised using a kind of rebstricted endonucleas such as HindIII, BamHI and the like, and cloned into the Agroacterium binary vector which has the same single slicing site as the chimeric gene to form a final construct. In the invention, pBI121, one kind of the Agroacterium binary vector, is preferably used to form the final construct.

In the invention, the final constructs are transformed into a plant. The plant is preferably selected from Tobacco and Arabidopsis thaliana. In the case of Arabidopsis, flowering buds cells are transformed by vacuum infiltration through an Agrobacterium system. Resulting seeds can carry foreign genes. Transgenic plants are regenerated from the seeds for Arabidopsis thaliana. In the case of Tobacco, plant cells are transformed by an Agrobacterium system through infection of leaf disc explants. Transgenic plants may be regenerated from the calluses carrying foreign genes.

The test for plant genome integration may be identified by Southern analysis. Expression at MRNA level may be confirmed by Northern blot. Then, expression at protein level can be confirmed by Western blot.

The in vitro biological activity of target proteins such as hG-CSF produced in seeds of transgenic plants such as Arabidopsis may be determined in a cell proliferation assay by MTT assay, for example, using a factor-dependent murine myeloblastic cell line NFS-60.

The invention will be in details described by the following examples in connection with the drawings.

### EXAMPLE 1

### Construction of Chimeric Gene pTZ/Phas/His/EK/hG-CSF (Construct H)

Amplification of hG-CSF

A nucleotide sequence (525 bp) encoding hG-CSF mature peptide (FIG. 15) in pB/KS/hG-CSF was first amplified by PCR using two following specific primers 5′GCSF-1 and 3′GCSF as shown in FIG. 16, which introduced a 5′ NcoI site and a 3′ AccI site to hG-CSF gene for sub-cloning. A 50 μl PCR reaction mixture containing 40 ng pB/KS/hG-CSF as a DNA template, 1×Pfu buffer (Stratgene, USA), 0.2 mM dNTP, 0.5 μM 5′GCSF-1 primer, 0.5 μM 3′GCSF primer and 2.5 units of Pfu DNA polymerase (2.5 u/μl, Stratgene, USA) was prepared. The PCR condition was set as follows: 94° C. for 5 minutes, then 25 cycles at 94° C. for 30 seconds, 58° C. for 30 seconds and 72° C. for 30 seconds, followed by 1 cycle at 72° C. for 7 minutes.

Construction of Chimeric Gene pTZ/Phas/His/EK/hG-CSF (Construct H)

The PCR product was purified and then subjected to the reaction for A-tailing. A 10 μl reaction mixture containing 300 ng PCR product, 1×Taq DNA polymerase reaction buffer (Promega, USA), 2.5 mM MgCl₂, 0.2 mM dATP and 5 units of Taq DNA polymerase (5 u/μl, Promega, USA) was incubated at 70° C. for 2 hours. The A-tailed PCR product was first ligated to a pGEM®-T vector. hG-CSF gene was then excised from a pGEM®-T/hG-CSF using NcoI and NotI, and cloned into a pET/His/EK vector containing 6× consecutive histidine-tag and enterokinase (EK) site. The resulting plasmid was named pET/His/EK/hG-CSF. Then, the whole gene cassette was excised using AccI and cloned into a pTZ/Phas vector containing a phaseolin promoter and a phaseolin terminator, forming a plasmid of pTZ/Phas/His/EK/hG-CSF (FIG. 11).

### EXAMPLE 2

### Construction of pBK/Phas/SP/His/EK/hG-CSF (Construct SH)

The plasmid of pGEM®-T/hG-CSF was constructed as described above. Then, the target gene was excised from the plasmid using NcoI and NotI, and cloned into a pET/SP/His/EK vector containing a partial phaseolin signal peptide sequence, a histidine-tag and EK site, forming a plasmid of pET/SP/His/EK/hG-CSF. The whole gene cassette was excised using NdeI and AccI, and cloned into a pBK/Phas/SP vector containing a promoter, the remaining part of a phaseolin signal peptide sequence. The resulting plasmid was named pBK/Phas/SP/His/EK/hG-CSF (FIG. 12).

### EXAMPLE 3

### Construction of Chimeric Gene pBK/Phas/SP/hG-CSF (Construct S)

The nucleotide sequence (525 bp) encoding for the hG-CSF mature peptide in pB/KS/hG-CSF was first amplified by PCR using two specific primers 5′GCSF-2 and 3′GCSF as shown in FIG. 16, which introduced a 5′ NdeI site and a 3′ AccI site to the target gene for sub-cloning. A PCR reaction mixture (except containing a 5′GCSF-2 primer instead of 5′GCSF-1 primer) and the PCR condition was the same as Example 1. The PCR product was purified and then subjected to the reaction of A-tailing as in Example 2. The A-tailed PCR product was ligated to the pGEM®-T vector. Then the target gene was excised from the pGEM®-T/hG-CSF-NoHis using NdeI and AccI, and cloned into a pBK/Phas/SP vector containing the phaseolin promoter, the phaseolin signal peptide sequence and the terminator. The resulting plasmid was named pBK/Phas/SP/hG-CSF (FIG. 13).

### EXAMPLE 4

### Cloning of Chimeric Genes into Agrobacterium Binary Vector

Agrobacterium binary vector pBI121 that consists of the right border (RB) and left border (LB) of T-DNA, a neomycin phosphotransferase II (NPT II) selectable marker and β-glucurondiase (GUS) screenable marker genes is used in this Example. The three chimeric genes in plasmids pTZ/Phas/His/EK/hG-CSF, pBK/Phas/SP/His/EK/hG-CSF and pBK/Phas/SP/hG-CSF as prepared above were excised using HindIII, and cloned into the Agrobacterium binary vector pBI121 to form three final constructs. They were named pBI/Phas/His/EK/hG-CSF(H) pBI/Phas/SP/His/EK/hG-CSF(SH) and pBI/Phas/SP/hG-CSF(S), respectively, and were ready for plant transformation (FIG. 14).

### EXAMPLE 5

### Plant Transformation

The three chimeric genes as prepared in Example 5 were transformed into both tobacco and Arabidopsis through an Agrobacterium system. An aliquot (40 μl) of Agrobacterium competent cells thawed on ice. Then the competent cells were gently mixed with 1 μl of plasmid DNA (˜500 ng) and put on ice for 1 minute. The Gene Pulser apparatus (BioRad) was set as 25 μF, 2.5 kV and 600 ohms, and the cell-DNA mixture was transferred to a pre-chilled electroporation cuvette (Bio-Rad, U.S.A.) and shacked to the bottom of the cuvette without any bubbles. Then, an electric pulse was applied to the cuvette. After pulsing, 1 ml of SOC medium (2% Bacto tryptone, 0.5% Bacto yeast extract, 10 mM NaCl, 2.5 mM KCl, 10 mM MgCl₂, 10 mM MgSO₄ and 20 mM glucose) was added to the cuvette. The cells were quickly resuspended and transferred to a 5 ml polypropylene round-bottom tube (Falcon) with shaking at 28° C. for 2 hours. Then, 5 μl, 50 μl and the remaining cells were spread on LB plates supplemented with 50 mg/L rifampicin, 25 mg/L gentamycin and 50 mg/L kanamycin. The plates were incubated at 28° C. for 2 days to select the transformed Agrobacterium colonies.

In Arabidopsis, flowering buds cells were transformed by vacuum infiltration through an Agrobacterium tumefaciens-Ti plasmid system. Resulting seeds carry foreign genes and transgenic plants can be regenerated from the seeds.

In tobacco, plant cells were transformed by an Agrobacterium system through infection of leaf disc explants. Transgenic plants were regenerated from calli which carry foreign gene.

Assay

Southern Blot Analysis

The test for genome integration was identified by Southern analysis. Genomic DNA was first extracted from transgenic tobacco or Arabidopsis by using the CTAB protocol of Doyle et al. (Doyle, J. D., Doyle, J. L. and Bailey, L. H. 1990, Isolation of plant DNA from fresh tissue, Focus 12, 13-15.1990). Genomic DNA (10 μg) was digested overnight with HindIII at 37° C. Then the digested DNA was separated by gel electrophoresis in a 0.8% agarose/TAE gel and transferred to a positively charged nylon membrane (Boehringer Mannheim, Germany) using VacuGeneXL Vacuum Blotting System (Pharmacia Biotech, U.S.A.). A sense DIG-labeled DNA probe for the target protein mature peptide sequence was prepared by using the DIG DNA Labeling Kit (Boehringer Mannheim, Germany) via PCR amplification. Hybridization with the sense DIG-labeled DNA probe and detection using the Anti-Digoxigenin-AP (alkaline phosphatase) were preformed according to the method described in the DIG Nucleic Acid Detection Kit (Boehringer Mannheim, Germany).

As shown in FIGS. 3 and 7, all the three chimeric genes, H, SH, and S as prepared in Example 5, were detected in the Arabidopsis plant genome and in the tobacco plant genome, and the sequence is about 3 kb.

Northern Blot Analysis

Total RNA was extracted from Arabidopsis developing siliques. Total silique RNA (10 μg) was separated by gel electrophoresis in a 1% agarose/formaldehyde gel and transferred to a positively charged nylon membrane (Boehringer Mannheim, Germany) using capillary transfer method for overnight. An anti-sense DIG-labeled DNA probe for the target protein peptide sequence was prepared by using the DIG DNA Labeling Kit (Boehringer Mannheim, Germany) via PCR amplification. Hybridization with the anti-sense DIG-labeled DNA probe and detection using the Anti-Digoxigenin-AP (alkaline phosphatase) were preformed according to the method described in the DIG Nucleic Acid Detection Kit (Boehringer Mannheim, Germany).

As shown in FIGS. 4 and 8, expression at mRNA level was confirmed as the transcripts about 700 bp of the three foreign genes were detected in the developing seeds.

Western Blot Analysis

Before blotted, total seed protein (100 μg) extracted from mature transgenic seeds was separated by 16.5% tricine-SDS-PAGE. Then, a tricine-gel, without staining, was equilibrated in Dunn transfer buffer (10 mM NaHCO₃, 3 mM Na₂CO₃ and 0.02% SDS) for 20 minutes. At the same time, a piece of polyvinylidene difluoride (PVDF) membrane was first treated with 100% methanol for 1 minute and then equilibrated in a Dunn transfer buffer for 20 minutes. The protein in the tricine-gel was blotted onto PVDF membrane by using a Trans-blot electrophoretic transfer cell (Bio-Rad, USA). The transfer cell was filled with the Dunn transfer buffer and placed in an ice-bath. Electro-transfer was performed at 44V for 1 hour.

After electro-blotting, the membrane was subjected to immunodetection using an AURORA Western Blot Chemiluminescent Detection System (ICN, USA). The membrane was first placed in the Dunn transfer buffer for 15 minutes and then rinsed twice with 1×phosphate buffered saline (PBS) (58 mM Na₂HPO₄, 17 mM NaH₂PO₄.2H₂O and 68 mM NaCl) for 5 minutes. The membrane was incubated in a blocking buffer (1×PBS, 0.2% Aurora TM blocking reagent and 0.1% Tween-20) for 1 hour and then for another hour in a blocking buffer containing 0.2 μg/ml anti-hG-CSF polyclonal antibody (R&D Systems Inc., USA) Unbound primary antibody was removed by washing the membrane in the blocking buffer for 5 minutes (2 times). Then the membrane was incubated in the blocking buffer with 1:5000 anti-goat IgG secondary antibody-alkaline phosphatase conjugate for 1 hour. Again, the unbound secondary antibody was removed by washing the membrane in the blocking buffer for 5 minutes (3 times). Then the membrane was washed in assay buffer [20 mM Tris-HCl (pH9.8), 1 mM MgCl₂] for 2 minutes (2 times). After adding 1 ml of a chemiluminescent substrate solution, the membrane was ready for film exposure and development.

As shown in FIGS. 5 and 9, expression at protein level was confirmed by Western blot with anti-hG-CSF polyclonal antibody. hG-CSF was detected in the mature transgenic seeds, all with expected molecular weights, protein from construct H is 20.5 KD, protein from construct SH is 21 KD, protein from construct S is 18.6 KD. The said proteins are all at a level of 0.2% compared to total extractable seed protein, or 200 μg hG-CSF per gram of seeds.

Functional Analysis

The in vitro biological activity of hG-CSF produced in seeds of transgenic Arabidopsis was determined in a cell proliferation assay using a factor-dependent murine myeloblastic cell line NFS-60. NFS-60 cells were totally dependent on interleukin-3 (IL-3) or macrophage colony-stimulating factor (M-CSF) for growth and maintenance of their viability in vitro. These cells also proliferate in response to hG-CSF. Therefore, NFS-60 cells were used in this functional analysis.

The cryovial containing about 1 ml NFS-60 cells (ATCC®, USA) was quickly retrieved from the liquid nitrogen storage tank and incubated in a 37° C. water bath with regular agitation to thaw the cells. Then, all cells from the cryovial were transferred into a 15 ml centrifuge tube. About 15 ml complete RPMI 1640 medium [16.2 g/L RPMI 1640 powder (Gibco, USA), 10 mM HEPES, 1 mM sodium pyruvate, 1.5 g/L sodium bicarbonate, 0.05 mM β-mercaptoethanol, 5 ng/ml human recombinant macrophage colony stimulating factor (M-CSF) (PeproTech, USA), 10% fetal bovine serum (FBS) and 1% PSN (50 μg/ml penicillin G sodium salt, 50 μg/ml streptomycin sulfate and 100 μg/ml neomycin sulfate)] was added to the cells drop wise with regular agitation to dilute the cryopreservative (DMSO) and prevented from the sudden change of osmolarity in cells. Then the cell mixture was centrifuged at 1000 rpm for 10 minutes at 20° C. The cell pellet was resuspended in 5 ml pre-warmed complete RPMI 1640 medium. Ten μl cells mixed with 10 μl trypan blue, which only stained non-viable cells in blue color, were then transferred onto a haemocytometer. The concentration and viability of the cells were determined by counting and observing the cells under microscope. Suitable amount of cells was seeded to the fresh complete RPMI 1640 medium in a culture flask so that the initial cell density was 2.5×10⁴ cells/ml. The cells were then allowed to grow at 37° C. in a 5% CO₂ incubator followed by every 2-3 days passage as the maximum cell density should not exceed 5×10⁵ cells/ml.

MTT Assay

A rapid colorimetric assay (MTT assay) (Mosmann, T. 1983, Rapid colorimetric assay for cellular growth and survival: Application to proliferation and cytotoxic assays, J. Immunol. Mehtods 65, 55-63) was performed to determine the proliferation of NFS-60 cells which was induced by the hG-CSF produced in transgenic Arabidopsis or tobacco.

Tetrazolium salt MTT (3-(4,5-dimethylthiazol-2-yl)-2,5-diphenyl tetrazolium bromide) assay was used for determination of NFS-60 cell proliferation induced by hG-CSF expressed in transgenic seeds. NFS-60 cells were first incubated in a culture flask until the cell density was about 3×10⁵ cells/ml. Then the cells were transferred to a 50 ml tube and centrifuged at 1500 rpm for 5 minutes. The supernatant was discarded and the cell pellet was resuspended in a suitable volume of RPMI 1640 medium [complete RPMI 1640 medium, without 10% FBS and 5 ng/ml human M-CSF (PeproTech, USA)] so that the cell density was 1×10⁵ cells/ml. One-hundred μl cell culture (˜10000 cells) was added to each well of a 96-well microplate and starved for 4-6 hours at 37° C. in a 5% CO₂ incubator. Then 100 μl of serially dilution of total seed protein extract (from transgenic seeds) in RPMI 1640 medium (complete RPMI 1640 medium, without 10% FBS and 5 ng/ml human M-CSF, but with 20% heat inactivated FBS) was added to each well triplicate. At the same time, 100 μl of serially dilution of purified rhG-CSF produced from E. coli (PeproTech, USA) was added to each well triplicate as a standard curve. The cells were then incubated at 37° C. for 48 hours. Twenty μl of MTT solution (5 mg/ml MTT in HPBS, pH 7.4) was added to each well and then incubated for 2 hours at 37° C. The plate was centrifuged at 2000 rpm for 10 minutes. All supernatant was removed and 100 μl DMSO was added to each well to break down the cells and dissolved the purple crystals. The intensity of purple color in each well was measured by using a Microplate Spectrophometer at OD₅₇₀.

As shown in FIGS. 6 and 10, the biological activity of hG-CSF expressed in the transgenic Arabidopsis and tobacco seeds were assayed, using the murine myeloblastic cell line BFS-60 and the MTT [3-(4,5-dimethylthiazol-2-yl)-2,5-diphenyltetrazolium bromide] method and purified hG-CSF as standard. The in vitro biological activity of the transgenic hG-CSF in the crude protein extract reached 70% of the pure hG-CSF standard.

The level of current hG-CSF expression (0.2% as total extractable seed protein) is comparable to and better than several other human proteins expressed in plants. For clinical application, a dose of 1 to 60 g of h-GCSF/kg body wt/day is required. A patient of 50 kg body wt thus requires 50 to 300 μg recombinant hG-CSF per day, equivalent to some 0.3 to 20 g of the transgenic seeds currently produced in this project.

The plant production method of the invention thus has a promising potential to mass-produce some of the most expensive biopharmaceuticals of restricted availability in a much cheaper way, which has high economic value for disease therapy, diagnosis and prevention, and is more accessible to the less affluent countries.

1. A recombinant construct for transforming a plant comprising a DNA sequence encoding a recombinant human cytokine and a promoter capable of directing the expression of said recombinant human cytokine in said plant.

2. A recombinant construct of claim 1, wherein said human cytokine is a human granulocyte colony stimulating factor (hG-CSF).

3. A recombinant construct of claim 2, wherein said human granulocyte colony stimulating factor is SEQ ID No. 1.

4. A recombinant construct of claim 1, wherein said promoter is a plant seed-specific promoter.

5. A recombinant construct of claim 2, wherein said promoter is a plant seed-specific promoter.

6. A recombinant construct of claim 3, wherein said promoter is a plant seed-specific promoter.

7. A recombinant construct of claim 4, wherein said plant seed-specific promoter is a phaseolin promoter.

8. A recombinant construct of claim 5, wherein said plant seed-specific promoter is a phaseolin promoter.

9. A recombinant construct of claim 6, wherein said plant seed-specific promoter is a phaseolin promoter.

10. A recombinant construct of claim 1 further comprising a sequence tag and a cleavage site.

11. A recombinant construct of claim 2 further comprising a sequence tag and a cleavage site.

12. A recombinant construct of claim 3 further comprising a sequence tag and a cleavage site.

13. A recombinant construct of claim 4 further comprising a sequence tag and a cleavage site.

14. A recombinant construct of claim 5 further comprising a sequence tag and a cleavage site.

15. A recombinant construct of claim 6 further comprising a sequence tag and a cleavage site.

16. A recombinant construct of claim 7 further comprising a sequence tag and a cleavage site.

17. A recombinant construct of claim 8 further comprising a sequence tag and a cleavage site.

18. A recombinant construct of claim 9 further comprising a sequence tag and a cleavage site.

19. A recombinant construct of claim 10, wherein said a sequence tag and a cleavage site comprises a His-tag and an EK.

20. A recombinant construct of claim 11, wherein said a sequence tag and a cleavage site comprises a His-tag and an EK.

21. A recombinant construct of claim 12, wherein said a sequence tag and a cleavage site comprises a His-tag and an EK.

22. A recombinant construct of claim 13, wherein said a sequence tag and a cleavage site comprises a His-tag and an EK.

23. A recombinant construct of claim 14, wherein said a sequence tag and a cleavage site comprises a His-tag and an EK.

24. A recombinant construct of claim 15 wherein said a sequence tag and a cleavage site comprises a His-tag and an EK.

25. A recombinant construct of claim 16, wherein said a sequence tag and a cleavage site comprises a His-tag and an EK.

26. A recombinant construct of claim 17, wherein said a sequence tag and a cleavage site comprises a His-tag and an EK.

27. A recombinant construct of claim 18, wherein said a sequence tag and a cleavage site comprises a His-tag and an EK.

28. A recombinant construct of claim 1 further comprising a phaseolin signal peptide.

29. A recombinant construct of claim 2 further comprising a phaseolin signal peptide.

30. A recombinant construct of claim 3 further comprising a phaseolin signal peptide.

31. A recombinant construct of claim 4 further comprising a phaseolin signal peptide.

32. A recombinant construct of claim 9 further comprising a phaseolin signal peptide.

33. A recombinant construct of claim 10 further comprising a phaseolin signal peptide.

34. A recombinant construct of claim 18 further comprising a phaseolin signal peptide.

35. A recombinant construct of claim 19 further comprising a phaseolin signal peptide.

36. A method for constructing a transgenic plant, comprising the steps of:
a) transforming a plant cell with a recombinant construct of claim 1; and
b) regenerating the transgenic plant from the plant cell to produce a recombinant human cytokine in seeds of said transgenic plant.

37. A method of claim 36, wherein said plant cell is transformed by an Agrobacterium system.

38. A method of claim 37, wherein said Agrobacterium system is an Agrobacterium tumefaciens-Ti plasmid system.

39. A method of claim 36, wherein said plant is selected from Arabidopsis thaliana and tobacco.

40. A method of claim 37, wherein said plant is selected from Arabidopsis thaliana and tobacco.

41. A method of claim 38, wherein said plant is selected from Arabidopsis thaliana and tobacco.

42. A method of claim 36, wherein said plant cell is transformed by vacuum infiltration of flowering buds for Arabidopsis thaliana or by infection of leaf disc explants for tobacco.

43. A method of claim 37, wherein said transgenic plant is regenerated from seeds for Arabidopsis thaliana or from calluses for tobacco.

44. A method of claim 38, wherein said transgenic plant is regenerated from seeds for Arabidopsis thaliana or from calluses for tobacco.

45. A method of claim 36 further comprising the step of cloning said recombinant construct with a plasmid vector pBI121.

46. A protein comprising a human cytokine.

47. A protein of claim 46, wherein said human cytokine is a human granulocyte colony stimulating factor.

48. A transgenic plant comprising a protein of claim 47.

49. A transgenic plant of claim 48, wherein said transgenic plant is selected from Arabidopsis thaliana and tobacco.

50. A seed of a transgenic plant of claim 48.


SEQUENCE LISTING (4 sequences)

SEQ ID NO: 1 - DNA, 525 bp, Homo sapiens
acccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctga

SEQ ID NO: 2 - DNA, 27 bp, Artificial Sequence
  Primer for PCR
gcagccatggccacccccctgggccct

SEQ ID NO: 3 - DNA, 26 bp, Artificial Sequence
  Primer for PCR
cgccatatgccacccccctgggccct

SEQ ID NO: 4 - DNA, 29 bp, Artificial Sequence
  Primer for PCR
gaagtatactcagggctgggcaaggtggc